(19) United States
(12) Patent Application Publication
(10) Pub. No.: US 2018/0279960 A1
(43) Pub. Date: Oct. 4, 2018

(54) METHOD AND APPARATUS FOR REAL-TIME DISCRIMINATIVE OCULAR ARTEFACT REMOVAL FROM EEG SIGNALS

(71) Applicant: Agency for Science, Technology and Research, Singapore (SG)

(72) Inventors: Xinyang Li, Singapore (SG); Cuntai Guan, Singapore (SG); Haihong Zhang, Singapore (SG); Kai Keng Ang, Singapore (SG); Zhuo Zhang, Singapore (SG)

(21) Appl. No.: 15/940,884

(22) Filed: Mar. 29, 2018

Related U.S. Application Data
(60) Provisional application No. 62/480,146, filed on Mar. 31, 2017.

(51) Int. Cl. A61B 5/00; A61B 5/04; A61B 5/0476

(52) U.S. Cl. CPC A61B 5/7207; A61B 5/0476; A61B 5/04012

(57) ABSTRACT

There is provided a method and apparatus for real-time discriminative ocular artefact removal from EEG signals. This is facilitated by integrating inter-class dissimilarity and within-class similarity in a regularized framework based on oscillatory correlation. Correspondingly, components related to ocular movements are extracted from the raw data as pseudo-artefact channels so that it is applicable to single-channel EEG data without a dedicated EOG or eye-tracker.

### CROSS REFERENCE TO RELATED APPLICATIONS

This application claims priority to U.S. Provisional Application No. 62/480,146, filed Mar. 31, 2017, the contents of which is hereby incorporated by reference in its entirety.

### FIELD OF INVENTION

The present invention relates to ocular artefact removal from EEG signals.

BACKGROUND

EEG-based inventions are experiencing rapid growth over a broad range of applications, such as, for example, brain computer interfaces (BCIs) for motor rehabilitation/cognitive training, monitoring of mental conditions and so forth.

The EEG is configured to record cerebral activities, but unfortunately, it also records electrical activities which do not originate from brain activity. These non-cerebral activities are known as artefacts, and can originate physiologically (generated by the patient body or muscle other than the brain) or extra-physiologically (generated from the environment). Artefact removal is critical for better accuracy and is especially important with dry EEG sensors which have shown great potential for use in home-based EEG applications. This is because dry sensors are more susceptible to signal contamination compared to wet sensors.

Algorithms derived for recovering artefact-free signals have been subject to extensive research. Amongst various possible artefacts, electrooculogram (EOG) artefacts induced by ocular movements such as the blinking of eyes are perceived in a unique manner, the ocular movements typically carries useful information stemming from cerebral activities.

Some methods for ocular artefact removal from EEG signals which are currently available are:

Methods with Extract Management

To capture eye movement information, eye movement correction procedure (EMCP) uses EOG recorded along with EEG, and subsequently, subtracts the EOG components from EEG after scaling based on regression. However, as the EOG may typically also contain components from brain activities, such subtraction based on regression generally causes a loss of relevant EEG signals. In addition to an EOG, a combination eye tracker and a frontal EEG are also used to capture ocular movements.

Methods Requiring Multiple Channels

For high-dimensional EEG data, independent component analysis (ICA) has been employed for eliminating ocular artefact components in EEG. By assuming that the observed EEG signal is a mixture of multiple unknown and mutually statistically independent sources, ICA solves the inverse problem and estimates the sources. Then, the source components corresponding to eye movement are identified and removed either manually or automatically using prior knowledge about the spatial pattern of the ocular artifacts. In ICA-based analysis, EOG is not necessary, while it is desirable to record sufficient EEG channels to capture as many sources as possible. Usually, ICA is applied to data sets recorded from at least ten EEG channels. And it is found that as few as thirty five channels are needed for source estimation in the study of concurrent locomotor and cognitive tasks. Although the requirement of the minimum number of channels may vary in different experiment tasks, the source separation would be less suitable when only a few EEG channels are available.

Methods Based on a Single Channel

In practical BCI systems, the number of available channels can be limited for the comfort and convenience of subjects/patients, and in some instances, there is only one channel of EEG in certain BCI systems. Thus, methods recovering artefact-free signals for a single-channel signal have been proposed. A multi-channel signal is obtained using time-delayed coordinates of the single-channel signal, and a standard ICA-based artefact correction follows. Signals with different time delays are also used in conventional approach, and a local singular spectrum analysis based on principal component analysis (PCA) is applied to the data matrix for artefact removal. In the ensemble empirical-mode decomposition ICA, intrinsic-mode functions (IMFs) of single-channel signal are obtained by empirical mode decomposition (EMD), and used as multi-channel data. Similarly, there are methods decomposing a single-channel signal into multiple components using wavelet decomposition, followed by standard source separation methods such as ICA.

Issues with Current Methods

For ocular artefact removal in BCI, the most challenging issue is to remove the artefacts with the minimal loss of the cerebral information. For instance, discriminative information of different motor imagery classes should remain intact after ocular artefact removal for the sake of classification. However, the aforementioned artefact removal algorithms are designed to be a pre-processing procedures, which is independent of the following classification or detection in BCI. The issue of accuracy drop caused by the loss of discriminative information in EEG signals pre-processing step has not been addressed.

### SUMMARY

In a first aspect, there is provided a system for real-time discriminate ocular artefact removal from EEG signals, the system including at least one data processor configured to:
smoothen, at a signal smoothener, raw EEG signals;
calculate, at a peak amplitude calculator, peak amplitudes of smoothened EEG signals;
select, at a peak range selector, a peak range of the smoothened EEG signals;
form, at an artefact channel former, a pseudo artefact channel;
enable, at a discriminative learner, discriminative learning; and
remove, at an artefact remover, ocular artefacts from the raw EEG signals.

In a second aspect, there is provided a data processor implemented method for real-time discriminate ocular artefact removal from EEG signals, the method comprising:
smoothening, at a signal smoothener, raw EEG signals;
calculating, at a peak amplitude calculator, peak amplitudes of smoothened EEG signals;
selecting, at a peak range selector, a peak range of the smoothened EEG signals;
forming, at an artefact channel former, a pseudo artefact channel;
enabling, at a discriminative learner, discriminative learning; and
removing, at an artefact remover, ocular artefacts from the raw EEG signals.

In a final aspect, there is provided a non-transitory computer readable storage medium embodying thereon a program of computer readable instructions which, when executed by one or more processors of a signal processing device, cause the signal processing device to carry out a method for real-time discriminate ocular artefact removal from EEG signals, the method embodying the steps of:
smoothening, at a signal smoothener of the signal processing device, raw EEG signals;
calculating, at a peak amplitude calculator of the signal processing device, peak amplitudes of smoothened EEG signals;
selecting, at a peak range selector of the signal processing device, a peak range of the smoothened EEG signals;
forming, at an artefact channel former of the signal processing device, a pseudo artefact channel;
enabling, at a discriminative learner of the signal processing device, discriminative learning; and
removing, at an artefact remover of the signal processing device, ocular artefacts from the raw EEG signals.

It will be appreciated that the broad forms of the invention and their respective features can be used in conjunction, interchangeably and/or independently, and reference to separate broad forms is not intended to be limiting.

### DESCRIPTION OF FIGURES

A non-limiting example of the present invention will now be described with reference to the accompanying drawings, in which:

FIG. 1 is a flow chart of a first example for a method for real-time discriminative ocular artefact removal from EEG signals;

FIG. 2 is a schematic diagram showing a system for executing a method for real-time discriminative ocular artefact removal from EEG signals;

FIG. 3 is a schematic diagram of a computing system for implementing the method of FIG. 1 and for providing computing resources for the system of FIG. 2;

FIG. 4 is a flow chart of a second example for a method for real-time discriminative ocular artefact removal from EEG signals;

FIG. 5 shows examples of constructed artefact signals;

FIG. 6 shows an example of an algorithm for optimization of ocular artefact correction coefficients;

FIG. 7 shows examples of training data and test data; and

FIG. 8 is a table showing test classification results.

### DETAILED DESCRIPTION

Embodiments of the present invention provide a method and apparatus for real-time discriminative ocular artefact removal from EEG signals. This is facilitated by integrating inter-class dissimilarity and within-class similarity in a regularized framework based on oscillatory correlation. Correspondingly, components related to ocular movements are extracted from the raw data as pseudo-artefact channels so that it is applicable to single-channel EEG data without a dedicated EOG or eye-tracker. Typically, ocular artefacts are more sporadic and irregular than oscillatory modulation caused by mental activities, resulting in lower correlations between instances.

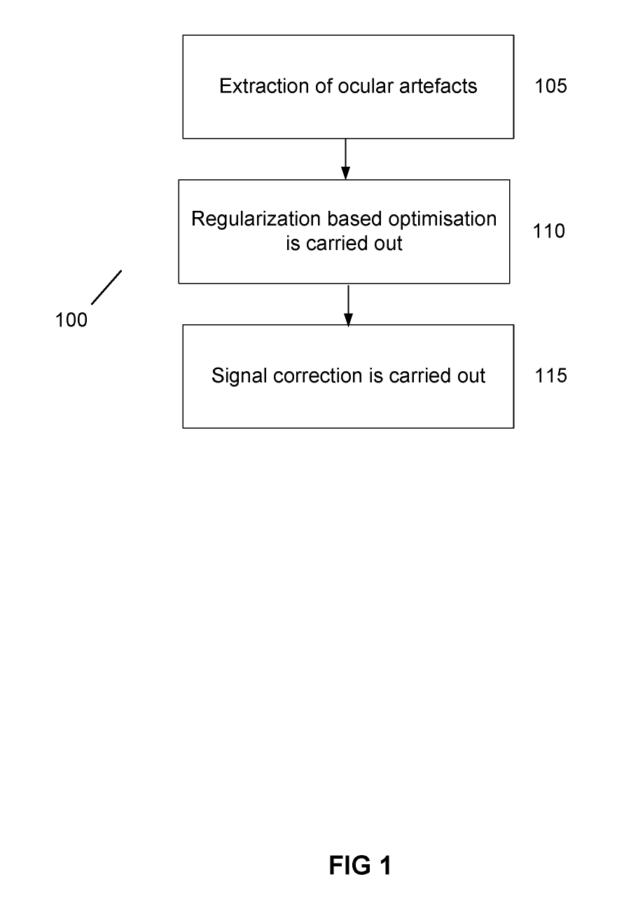

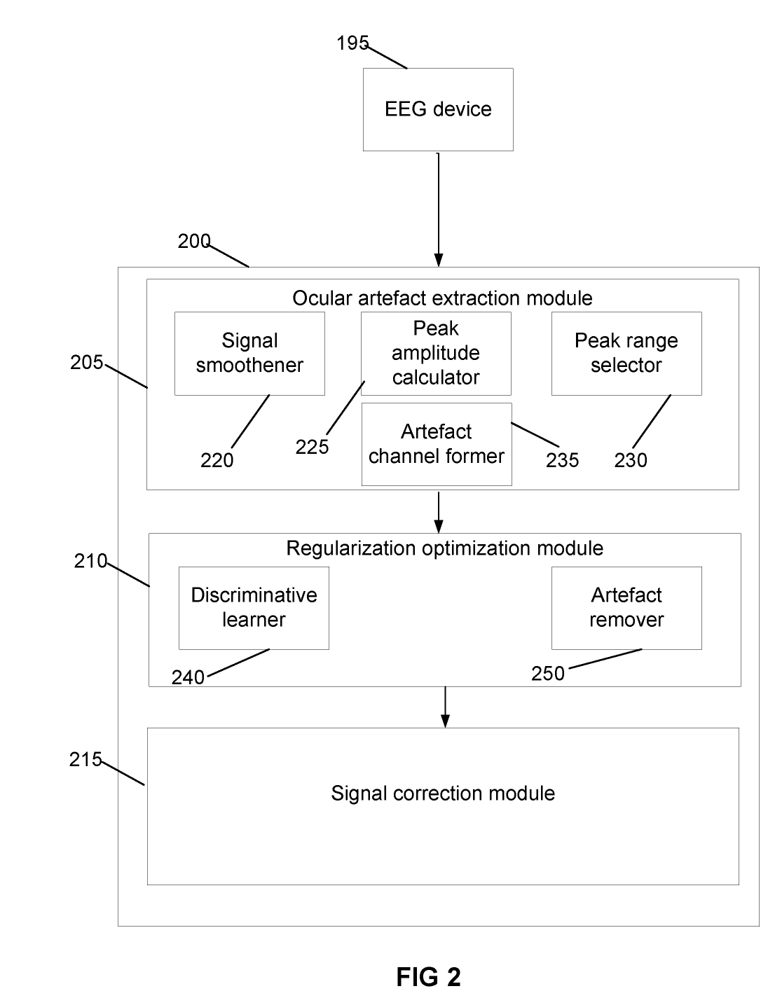

Referring to FIG. 1, there is shown a first example for a method 100 for real-time discriminative ocular artefact removal from EEG signals. In the first example, the method 100 shows a general overview of the method 100, and detailed breakdowns of the respective steps are provided in subsequent portions of the description. In addition, reference is also made to FIG. 2, which shows a system 200 for executing the method 100.

After a subject is set-up with the system 200 in a manner whereby the subject's EEG signals are able to be captured by an EEG device 195, at step 105, extraction of ocular artefacts is carried out. This can be carried out using an ocular artefact extraction module 205 of the system 200. It should be noted that the artefact extraction module 205 includes sub components which will be described in greater detail in a subsequent portion of the description.

At step 110, a regularization based optimisation is carried out. This can be carried out using a regularization optimisation module 210 of the system 200. It should be noted that the regularization optimisation module 210 includes sub components which will be described in greater detail in a subsequent portion of the description.

At step 115, a signal correction is carried out, and this can be carried out using a signal correction module 215 of the system 200. Processes carried out by the signal correction module 215 will also be described in a subsequent portion of the description.

The method 100 describes general broad steps which enables the real-time discriminative ocular artefact removal from EEG signals, which correspondingly provides EEG signals with minimal cerebral information loss.

Referring to FIG. 2, the ocular artefact extraction module 205 includes components such as, a signal smoothener 220, a peak amplitude calculator 225, a peak range selector 230, and an artefact channel former 235. It should be appreciated while the components 220, 225, 230, 235 are shown to be discrete components, the components can be a single or combined sub-modules configured to carry out respective tasks of the various components of the ocular artefact detection module 205. The EEG device 195 can include an EEG amplifier to amplify, and convert EEG signals.

The ocular artefact extraction is carried out on raw EEG data X₀(t) ∈ [symbol] ⁿ^{_{c}}^{×n}^{ₜ} where n_{c} is the number of channels and nₜ is the number of time samples. Given the analysis of the morphology characteristic of the eye movements related potentials, the moving average filter is applied to the raw EEG data to obtain the smoothed signal xₛ(t) at the signal smoothener 220 for further artefact extraction, as follows:

$${x}_{s} ( t ) = \frac{1}{m} {\sum }_{j = - \frac{m}{2}}^{j = \frac{m}{2}} {x}_{o} ( t + j ) \qquad ( 1 )$$

where m is the number of the neighboring points used in the moving average filter, and x₀(t) ∈ [symbol] ⁿ^{ₜ} is the EEG signal from one arbitrary channel, or in other words, one arbitrary row of X₀(t).

The relative amplitude of the peaks is calculated as

h(t)=max(|xₛ(t)−xₛ(t−1)|, |xₛ(t+1)−xₛ(t)|) (2)

One ocular artefact could include both positive and negative peaks. In other words, a peak could consist of an ocular artefact together with either the peak before it or the peak after it. To construct the artefact as completely as possible, in (2), a maximum relative amplitude is used as the measurement of the peak. This is determined at the peak amplitude calculator 225. By defining the peak amplitude range parameter hᵣ as

hᵣ=[h_{b}, hᵤ] (3)

Then, find the set [symbol] ₜ containing time indexes of those peaks with amplitude in the range hᵣ as

$${𝒫}_{t} = \{ {t}_{i} : \frac{m}{2} < {t}_{i} < {n}_{t} and {b}_{h} < h ( {t}_{i} ) < {h}_{u} \} \qquad ( 4 )$$

For each element tᵢ ∈ [symbol] ₜ, i=1, 2, . . . , | [symbol] ₜ, let tᵢ^{zb} and tᵢ^{za} be the nearest zero points before and after tᵢ, i.e,

$$\begin{matrix}\begin{matrix}{t}_{i}^{zb} - arg {max}_{t} t & s . t . & t < {t}_{i} and {x}_{s} ( t ) = 0\end{matrix} & ( 5 ) \\ \begin{matrix}{t}_{i}^{za} - arg { min}_{t} t & s . t . & t < {t}_{i} and {x}_{s} ( t ) = 0\end{matrix} & ( 6 )\end{matrix}$$

It is not likely to obtain a true zero point for real discrete signal. Thus, in practical implementation we set a small threshold, and signal points with absolute values below the threshold are regarded as zero points. This is carried out in the peak range selector 230. With the time period [tᵢ^{zb}, tᵢ^{za}] obtained for each peak point tᵢ ∈ [symbol] ₜ, the artefact signal xₐ(t) is constructed as

$${x}_{a} ( t ) = \{ \begin{matrix}{x}_{s} ( t ) , t ∈ [ {t}_{i}^{zb} , {t}_{i}^{za} ] with i = 1 , 2 , … , [ {𝒫}_{t} \vert ; & \\ 0 , else . & \end{matrix} \qquad ( 7 )$$

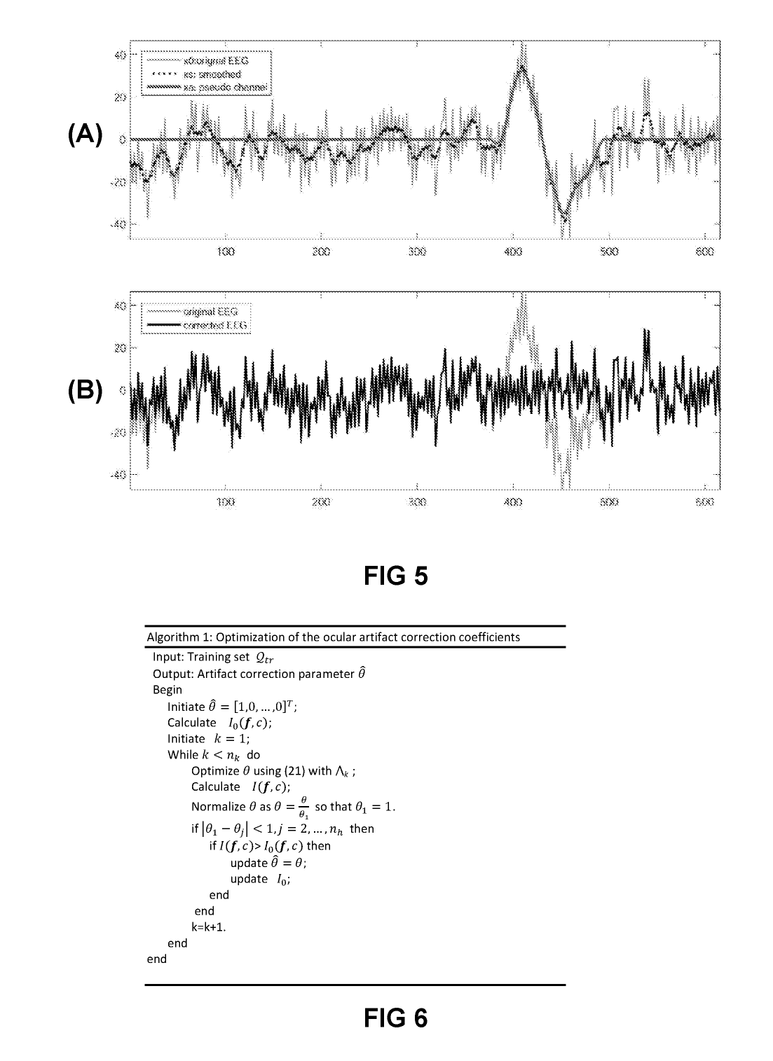

An example of constructing xₐ(t) from xₛ(t) is illustrated in FIG. 5. As shown by FIGS. 5(a) and 5(b), xₐ(t) is zero except those points belonging to/associated with peaks whose amplitudes are within a certain range. In this way, EEG data that is not contaminated with the ocular artefacts is not diminished after artefact correction.

Moreover, with different amplitude ranges hᵣ^{j}=[h_{b}^{j}, hᵤ^{j}]^{T}, j=1, 2, . . . , nₕ, xₐ^{j}(t) can be extracted correspondingly, where nₕ is the number of peak amplitude ranges.

At the artefact channel former 235, define xₐ(t) as the matrix containing all artefact signals xₐ^{j}(t), as follows:

$${X}_{a} ( t ) = [ \begin{matrix}{x}_{a}^{1} ( t ) \\ . \\ . \\ . \\ {x}_{a}^{{n}_{h}} ( t )\end{matrix} ] \qquad ( 8 )$$

Xₐ(t) in (8) can be regarded as the pseudo artefact channel. Using Xₐ(t) for artefact correction is even more advantageous than using the real EOG signal. As it is zero at most of the time points, it would cause less information loss when carrying out the artefact removal. Moreover, by separating the artefacts by using amplitudes of the peaks, artefacts corresponding to different ocular movements could be separated with different filtering parameters assigned. It is more flexible to maintain the discriminative information in EEG signals than a conventional Eye Movement Correction Procedure (EMCP), where one propagation factor is estimated for one EEG-EOG pair.

Similarly, the regularization optimization module 210 includes components such as, a discriminative learner 240, and an artefact remover 250. In some embodiments, the artefact remover 250 acts as a filter to remove pseudo artefact channels from raw EEG signals. The artefact remover 250 can include parameters defined in the discriminative learner 240. As noted earlier, while the components 240, 250 are shown to be discrete components, the components can be a single or combined sub-modules configured to carry out respective tasks of the various components of the regularization optimization module 210. The components can be implemented entirely by software to be executed on standard computing device hardware, which may comprise one hardware unit or different computer hardware units distributed over various locations.

In the regularization optimization module 210, let i be the trial index, and we define the signal after correction in the ocular artefact extraction module 205 as x_{c,i}(t), i.e.,

x_{c,i}(t)=x_{o,i}(t)−θₐ^{T}X_{a,i}(t) (9)

where θₐ^{T} ∈ [symbol] ⁿ^{ₕ} is the artefact correction coefficient or filtering coefficient, scaling the artefacts in EEG to be removed and similar to the propagation factor in the conventional EMCP.

As the oscillatory correlation is effective for source separation, the correction coefficient θₐis optimised using the oscillatory correlations between EEG trials, as ocular artefacts should be more sporadic and irregular compared to the oscillatory modulation caused by mental activities.

By rewriting (9) as

x_{c,i}(t)=θ^{T}Xᵢ(t) (10)

where

$$\begin{matrix}{X}_{i} ( t ) = [ \begin{matrix}{x}_{0 , i} ( t ) \\ {X}_{a , i} ( t )\end{matrix} ] & ( 11 ) \\ θ = [ \begin{matrix}1 \\ {θ}_{a}\end{matrix} ] & ( 12 )\end{matrix}$$

Define the instantaneous power of x_{c,i}(t) as ϕᵢ(t), i.e.,

ϕᵢ(t)={square root over ((θ^{T}Xᵢ(t))²+(θ^{T}Hᵢ(t))²)} (13)

where Hᵢ(t) is the Hilbert transform of Xᵢ(t). To obtain an average oscillatory correlation between multiple trials, for each trial i, the average instantaneous power for all trials except i is defined as ψᵢ(t), i.e.,

$${ψ}_{i} ( t ) ≡ \frac{1}{n - 1} {\sum }_{j \neq i} \sqrt{{( {θ}^{T} {X}_{j} ( t ) )}^{2} + {( {θ}^{T} {H}_{j} ( t ) )}^{2}} \qquad ( 14 )$$

Thus, the objective function maximizing the cross-trial oscillatory correlation is

$$\begin{matrix}\hat{θ} = {max}_{θ} \frac{1}{n} {\sum }_{i} {ρ}_{{φ}_{i}} ( t ) , {ψ}_{i} ( t ) & ( 15 ) \\ where & \\ {ρ}_{{φ}_{i ( t ) ,} {ψ}_{j} ( t )} = \frac{\frac{1}{{n}_{t}} \int \overline{{φ}_{l}} ( t ) \overline{{ψ}_{l}} ( t ) dt}{\frac{1}{{n}_{t}} \sqrt{\int {\overline{{φ}_{l}}}^{2} ( t ) dt \cdot \int {\overline{{ψ}_{l}}}^{2} ( t ) dt}} & ( 16 ) \\ With & \\ {\overline{φ}}_{l} = {φ}_{i} ( t ) - \frac{1}{{n}_{t}} \int {φ}_{i} ( t ) dt & ( 17 ) \\ \overline{{ψ}_{l}} = {ψ}_{i} ( t ) - \frac{1}{{n}_{t}} \int {ψ}_{i} ( t ) dt & ( 18 )\end{matrix}$$

Optimizing θ using (15) could maximize the average cross trial oscillatory correlation so that sporadic ocular artefacts could be subdued, but it is not enough to maintain the discriminative information. To ensure that the artefact correction could benefit the classification in a Brain Control Interface (BCI), θ should be learnt in a discriminative manner, which is different from the regressive coefficient estimation or source separation. This is carried out in the discriminative learner 240.

Thus, the interclass oscillatory correlation, rᵢ, is taken into consideration, which can be calculated as

$${r}_{i} = \frac{1}{\vert {Q}^{+} \vert \vert {Q}^{-} \vert} {\sum }_{i ∈ Q} + {\sum }_{j ∈ Q} - {ρ}_{{φ}_{i ( t ) ,} {ψ}_{j} ( t )} \qquad ( 19 )$$

where Q^{c} is the set of trial index belonging to class c, and |Q^{c}| is the number of the elements in Q^{c} with the class label c ∈ {+,−}. Similarly, the within-class oscillatory correlation for class c, r_{w}^{c}, can be calculated as

$${r}_{w}^{c} = \frac{1}{\vert {Q}^{c} \vert} {\sum }_{i ∈ {Q}^{c}} {ρ}_{{φ}_{i ( t ) ,} {ψ}_{i} ( t )} \qquad ( 20 )$$

For joint artefact correction and discriminative feature learning, a regularized oscillatory correlation objective function is carried out as

$$\hat{θ} = arg {max}_{θ} ( 1 - λ ) {\sum }_{c} {λ}_{C} {r}_{w}^{c} - λ {r}_{i} , with {\sum }_{c} {λ}^{C} = 1 \qquad ( 21 )$$

where λ^{C} is the weight given to the within-class oscillatory correlation for class c, and λ controls the weights of within-class and inter-class oscillatory correlation. The regularization method has been widely used in the computational model development in BCI to address within-class similarity and inter-class dissimilarity simultaneously. With (21), θ is optimized so that the within-class oscillatory correlation could be maximized while the inter-class oscillatory is minimized.

At the artefact remover 250, given the discriminative oscillatory correlation, two kinds of features are extracted, namely, the correlation feature and the power feature. The oscillatory correlation feature f_{r,i}^{c} can be obtained as

$${f}_{r , i}^{c} = \frac{1}{{t}_{2} - {t}_{1}} {\int }_{{t}_{1}}^{{t}_{2}} {ρ}_{{φ}_{i ( t ) ,} {\overline{Φ}}^{c} ( t )} dt \qquad ( 22 )$$

where Φ̅^{c}(t) is the average instantaneous power of class c, i.e.,

$${\overline{Φ}}^{c} ( t ) = \frac{1}{j ∈ {Q}^{c}} \sqrt{{( {θ}^{T} {X}_{j} ( t ) )}^{2} + {( {θ}^{T} {H}_{j} ( t ) )}^{2}} \qquad ( 23 )$$

Thus, for each trial and each time window, a pair of correlation features is extracted. The power feature f_{p,i} for trial i could be extracted as

$${f}_{p , i} = \frac{1}{{t}_{2} - {t}_{1}} {\int }_{{t}_{1}}^{{t}_{2}} {\vert \vert {x}_{c , i} ( t ) \vert \vert}^{2} dt \qquad ( 24 )$$

where [t₁, t₂] is the time window for the power calculation. With (21), for a certain time window, if the power of the signals from one class is high, that from the other class would be low, and vice versa. Therefore, the band power feature fₚ is consistent with the objective function. This can be carried out at the artefact remover 250.

Regarding selection of the regularization parameter in (21), mutual information between the feature f and class label c, i.e., I(f,c) is used, instead of cross validation to reduce the computational complexity. The mutual information has been widely used for feature optimization in BCI, and details of the calculation can be found in documents [1], and [2] as indicated in the references section of the present document.

Let ∧ₖ=[λ, λ⁺, λ⁻], k ∈ {1, 2, . . . , nₖ}, which contains all nₖ combinations of regularization parameters, e.g., ∧₁=[0,0.5,0.5], ∧₂=[0.1,0.5,0.5], etc. With different ∧ₖ, using (21), followed by the calculation of feature f and the mutual information I(f,c). Given I(f,c) calculated based on different ∧ₖ, a θ which yields the highest mutual information I(f,c) is selected. By introducing (f,c), a desired combination of the regularization term ∧ₖ is obtained. Furthermore, feature discrimination is enhanced during the optimization.

To ensure that the artefacts are not enhanced, an additional constraint for the optimization of θ, which is

|θ₁−θⱼ|<1, j−2, . . . , nₕ (25)

Q1≡1 (26)

where θⱼ is the j-th element of θ. θ₁ is the weight corresponding the raw EEG signal, x_{0,i}(t), which is constrained to be 1. With (25), θⱼ can't be positive, and subsequently, the detected artefact will not be enhanced.

By maximizing the inter-class oscillatory differences, the artefact correction parameter could also be driven toward increasing the amplitude of the ocular artefacts if the artefacts contribute to the discrimination between two classes. Although it could also be addressed by adding extra constraint terms accepting the solutions that suppress the ocular artefacts. The suppression of the ocular artefacts is carried out by the signal correction module 215.

Referring to FIG. 6, further details of the optimization process in the regularization optimization module 210 are described in Algorithm 1. It should be noted that Algorithm 1 is merely illustrative.

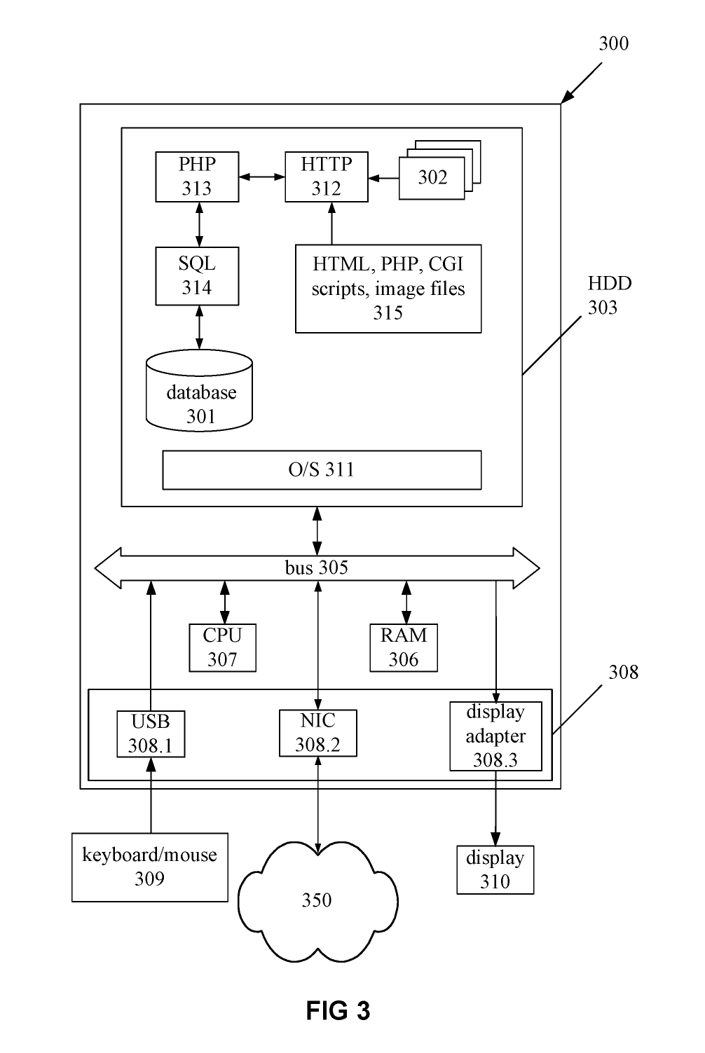

Referring to FIG. 3, there is shown a computing system 300 which can be configured to carry out the method 100, and can be used to provide the computing resources for the system 200.

The computing system 300 is able to communicate with other processing devices, as required, over a communications network 350 using standard communication protocols.

Components of the computing system 300 can be configured in a variety of ways. The components can be implemented entirely by software to be executed on standard computer server hardware, which may comprise one hardware unit or different computer hardware units distributed over various locations, some of which may require the communications network 350 for communication. A number of the components or parts thereof may also be implemented by application specific integrated circuits (ASICs) or field programmable gate arrays.

In the example shown in FIG. 3, the computing system 300 is a commercially available computer system based on a 32 bit or a 64 bit Intel architecture, and the processes and/or methods executed or performed by the computing system 300 are implemented in the form of programming instructions of one or more software components or modules 302 stored on non-volatile (e.g., hard disk) computer-readable storage 303 associated with the computing system 300. At least parts of the software modules 302 could alternatively be implemented as one or more dedicated hardware components, such as application-specific integrated circuits (ASICs) and/or field programmable gate arrays (FPGAs).

The computing system 300 includes at least one or more of the following standard, commercially available, computer components, all interconnected by a bus 305:

1. random access memory (RAM) 306;

2. at least one computer processor 307, and

3. external computer interfaces 308:

a. universal serial bus (USB) interfaces 308.1 (at least one of which is connected to one or more user-interface devices, such as a keyboard, a pointing device (e.g., a mouse 309 or touchpad),

b. a network interface connector (NIC) 308.2 which connects the computing system 300 to a data communications network 350; and

c. a display adapter 308.3, which is connected to a display device 310 such as a liquid-crystal display (LCD) panel device.

The computing system 300 can also include a plurality of standard software modules, including:

1. an operating system (OS) 311 (e.g., Linux or Microsoft Windows);

2. web server software 312 (e.g., Apache, available at http://www.apache.org);

3. scripting language modules 313 (e.g., personal home page or PHP, available at http://www.php.net, or Microsoft ASP); and

4. structured query language (SQL) modules 314 (e.g., MySQL, available from http://www.mysql.com), which allow data to be stored in and retrieved/accessed from an SQL database.

Together, the web server 312, scripting language 313, and SQL modules 314 provide the computing system 300 with the general ability to allow users of the network 350 with standard computing devices equipped with standard web browser software to access the computing system 300 and in particular to provide data to and receive data from the database 301. It will be understood by those skilled in the art that the specific functionality provided by the computing system 300 to such users is provided by scripts accessible by the web server 312, including the one or more software modules 302 implementing the processes performed by the computing system 300, and also any other scripts and supporting data 315, including markup language (e.g., HTML, XML) scripts, PHP (or ASP), and/or CGI scripts, image files, style sheets, and the like.

The boundaries between the modules and components in the software modules 302 are exemplary, and alternative embodiments may merge modules or impose an alternative decomposition of functionality of modules. For example, the modules discussed herein may be decomposed into submodules to be executed as multiple computer processes, and, optionally, on multiple computers. Moreover, alternative embodiments may combine multiple instances of a particular module or submodule. Furthermore, the operations may be combined or the functionality of the operations may be distributed in additional operations in accordance with the invention. Alternatively, such actions may be embodied in the structure of circuitry that implements such functionality, such as the micro-code of a complex instruction set computer (CISC), firmware programmed into programmable or erasable/programmable devices, the configuration of a field-programmable gate array (FPGA), the design of a gate array or full-custom application-specific integrated circuit (ASIC), or the like.

Each of the steps of processes (for example, the method 100) performed by the computing system 300 may be executed by a module (of software modules 302) or a portion of a module. The processes may be embodied in a non-transient machine-readable and/or computer-readable medium for configuring a computer system to execute the method. The software modules 302 may be stored within and/or transmitted to a computer system memory to configure the computer system to perform the functions of the module.

The computing system 300 normally processes information according to a program (a list of internally stored instructions such as a particular application program and/or an operating system) and produces resultant output information via input/output (I/O) devices 308. A computer process typically includes an executing (running) program or portion of a program, current program values and state information, and the resources used by the operating system to manage the execution of the process. A parent process may spawn other, child processes to help perform the overall functionality of the parent process. Because the parent process specifically spawns the child processes to perform a portion of the overall functionality of the parent process, the functions performed by child processes (and grandchild processes, etc.) may sometimes be described as being performed by the parent process.

Experiment Setup

Some embodiments of the method were tested using an experimental setup which will be described in the following paragraphs.

EEG data from a subject were obtained using a Neurosky dry EEG headband with one bipolar channel, which was positioned at a frontal site Fp1. The sampling rate was 256 Hz. Sixty eight subjects participated in the experiment, and for each subject, three sessions of a Color Stroop test were recorded. In each session, there were forty Stroop trials, during which each subject was assumed to be concentrating on the test. Each Stroop trial was followed by an idle period when the subject could relax. The Stroop trial lasted around ten seconds while the idle period between two Stroop trials was around fifteen seconds. To increase the number of trials, a four second window with a window-shift of two seconds was applied to segment EEG data recorded during Stroop trials, which yielded data of the attention class.

The same segmentation was also applied to EEG recorded during idle periods, which yielded data of the idle class. Only the segments at the beginning of the idle periods were used so that the final data set is balanced between the two classes, i.e., the attention class and idle class. Moreover, the first and second half of the original Stroop and idle trials were truncated into training trials and test trials, respectively. In this way, the test set was totally independent of the training set. For each subject, the number of total truncated trials was around two hundred and forty.

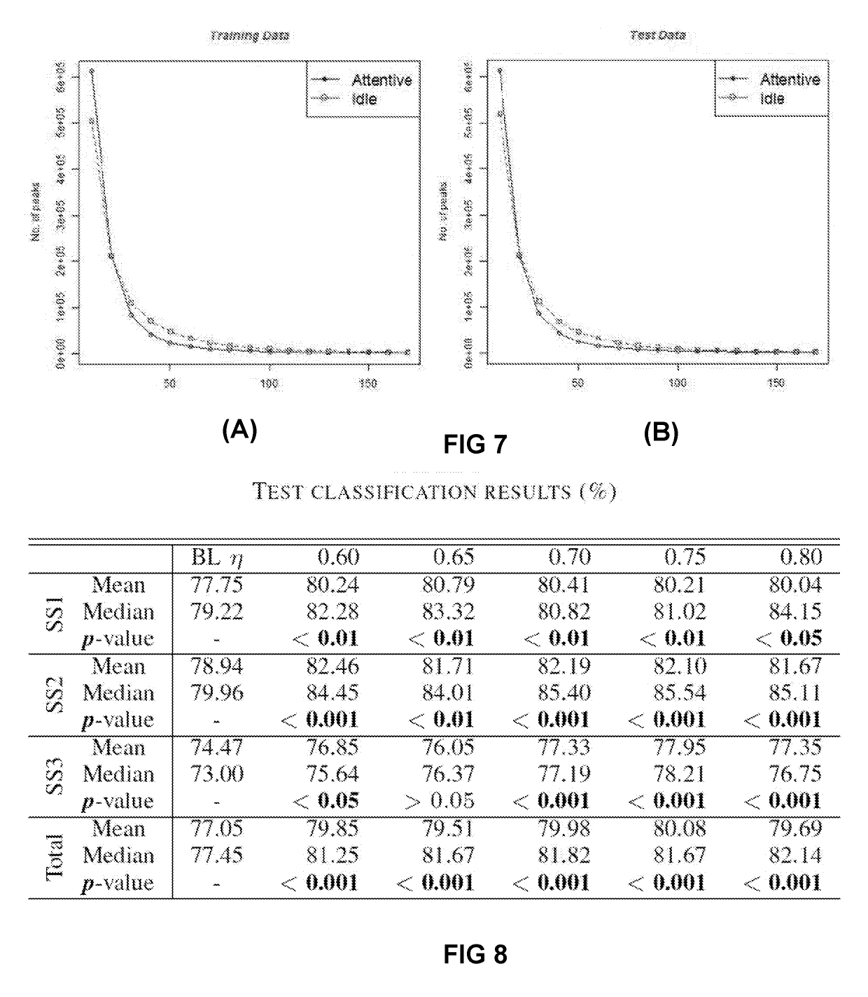

The experiment was focussed on the ocular artefacts during attention detection as ocular movements are closely related to attentive states. Thus, whether a subject is attentive or concentrating is typically reflected by the subject's ocular movements. In FIG. 7, the number of peaks in different hᵣ (peak amplitude ranges) are compared between attentive and idle states. Seventeen peak amplitude bins ranging from 10 to 170 are investigated with the width of each bin as 10. The x-axis in FIG. 7 represents the beginning of each bin while the y-axis represents the sum of the number of peaks in the amplitude range averaging across all the subjects for three sessions. It can be observed that for both training and test sets, the peaks of idle state are consistently more than that of the attentive state in the amplitude range of around 30-80.

In the experiment, raw EEG data is smoothened with m=10 in (1), and the number of the amplitude ranges is 2, i.e. nₕ=2, yielding Xₐ(t)∈ R^{2×n}^{ₜ} in (8). For each trial, the threshold to find the zero points t^{za} and t^{zb} is twice of the minimal absolute value of xₛ(t) for the trial. The regularization parameters λ and λ^{c} are pre-set to be in the range [0,0.1, . . . 0.5], yielding a total of thirty six combinations contained in ∧ with nₖ=36. (21) is optimized by limited-memory Broyden-Fletcher-Goldfarb-Shanno (L-BFGS) (implemented using MATLAB's minFunc). After the ocular artefact correction, a filter-bank containing 9 frequency bands (2-6 Hz, 6-10 Hz, . . . , 34-38 Hz) is applied on x_{c}(t). For each frequency band, we calculate the features for every 2 s window with 1 s window overlapping. Mutual information is applied to select the best four features, and, subsequently, the selected features are classified into the attention class or the idle class by a linear discriminant analysis (LDA) classifier [6].

FIG. 8 summarizes the classification results of the proposed ocular artefact correction method (OAC) compared with the baseline method (BL) for which no artefact correction is applied. Results of Session 1, Session 2 and Session 3 are indicated by “SS1”, “SS2” and “SS3”, respectively. As shown in FIG. 8, for all three sessions the proposed method improves both the median and average classification accuracies, the significance of which is validated by paired t-test with almost all p-values below 0.05.

When evaluation is carried out on a real world EEG data set comprising sixty eight subjects performing cognitive tasks, the results show that the approach is capable of suppressing the artefact components but also improving the discriminative power of a classifier with statistical significance. Thus, the compounding issues induced by ocular movements in cognitive EEG study are minimised.

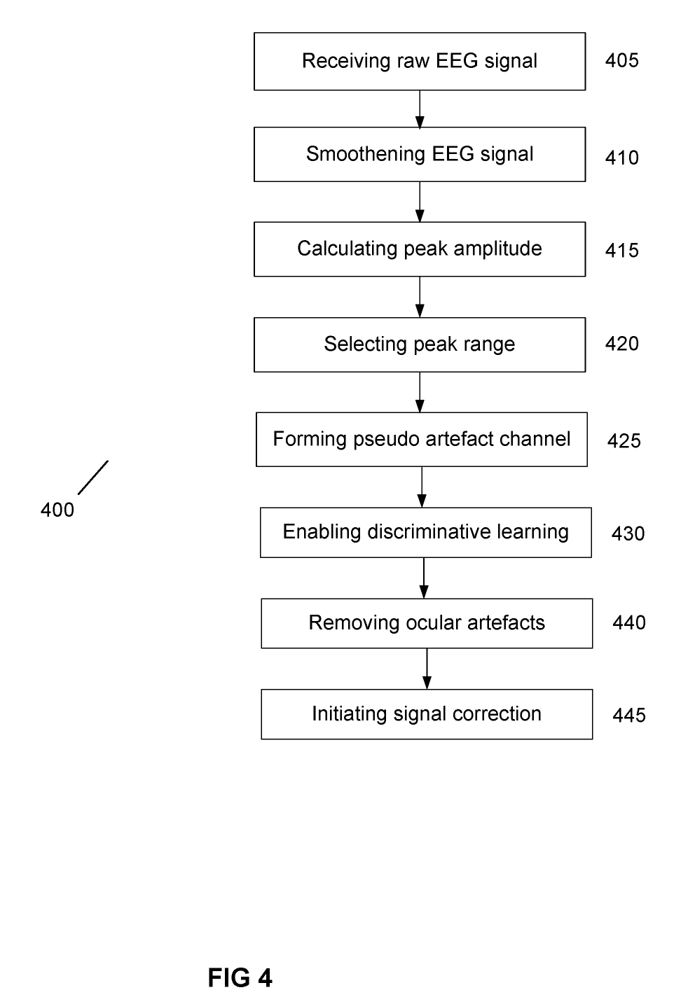

Referring to FIG. 4, there is shown a second example for a method 400 for real-time discriminative ocular artefact removal from EEG signals. In addition, reference is also made to FIG. 2, which shows the system 200 for executing the method 400. Generally, steps of the method 400 are shown to be carried out using components or sub-modules of the system 200, although it should be appreciated that the respective components or sub-modules need not be separate with each other.

At step 405, a subject's raw EEG signal is received. The subject's raw EEG signal can be obtained using the EEG device 195 used by the subject.

At step 410, the raw EEG signal undergoes smoothening, and this can be carried out at the signal smoothener 220 of the ocular artefact extraction module 205. Subsequently, peak amplitudes of the smoothened EEG signal are determined at step 415, and this can be carried out at the peak amplitude calculator 225.

At step 420, a peak range of the smoothened EEG signal is selected using the peak range selector 230. Once the peak range is selected, at step 425, a pseudo artefact channel is formed at the artefact channel former 235.

Subsequently, discriminative learning is enabled at step 430, and this is done at the discriminative learner 240 of the regularization optimization module 210. At the regularization optimization module 210, and ocular artefact removal from the raw EEG signals is carried out at step 440, at the artefact remover 250. In some embodiments, the artefact remover 250 acts as a filter to remove pseudo artefact channels from raw EEG signals. The artefact remover 250 can include parameters defined in the discriminative learner 240.

Finally, the signal correction for the raw EEG signals is initiated at step 445 using the signal correction module 215.

Thus, the method 400 describes steps which enable the real-time discriminative ocular artefact removal from EEG signals, which correspondingly provides EEG signals with minimal cerebral information loss as ocular artefacts are suppressed and not enhanced.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope of the invention.

### REFERENCES

[1]. K. K. Ang, Z. Y. Chin, C. Wang, C. Guan, and H. Zhang, “Filter bank common spatial pattern algorithm on BCI competition IV datasets 2a and 2b,” Frontiers in Neuroscience, vol. 6, no. 39, 2012.

[2]. K. K. Ang, Z. Y. Chin, H. Zhang, and C. Guan, “Mutual information based selection of optimal spatial-temporal patterns for single-trial EEG based BCIs,” Pattern Recognition, vol. 45, no. 6, pp. 2137-2144, 2012.

1. A system for real-time discriminate ocular artefact removal from EEG signals, the system including at least one data processor configured to:
smoothen, at a signal smoothener, raw EEG signals;
calculate, at a peak amplitude calculator, peak amplitudes of smoothened EEG signals;
select, at a peak range selector, a peak range of the smoothened EEG signals;
form, at an artefact channel former, a pseudo artefact channel;
enable, at a discriminative learner, discriminative learning; and
remove, at an artefact remover, ocular artefacts from the raw EEG signals.

2. The system of claim 1, the at least one data processor further configured to:
initiate, at a signal correction module, signal correction of the raw EEG signals.

3. The system of claim 1, wherein the signal smoothener, the peak amplitude calculator, the peak range selector and the artefact channel former are integrated in an ocular artefact extraction module.

4. The system of claim 1, wherein the discriminative learner, and the artefact remover are integrated in a regularization optimization module.

5. The system of claim 1, wherein the signal smoothener relies on a moving average filter.

6. The system of claim 1, wherein the peak amplitude calculator determines a maximum relative amplitude of peaks of the smoothened EEG signal.

7. The system of claim 6, wherein separation of the ocular artefacts using the maximum relative amplitude of peaks enables separation of different ocular movements.

8. The system of claim 1, wherein the discriminative learner uses oscillatory correlation.

9. A data processor implemented method for real-time discriminate ocular artefact removal from EEG signals, the method comprising:
smoothening, at a signal smoothener, raw EEG signals;
calculating, at a peak amplitude calculator, peak amplitudes of smoothened EEG signals;
selecting, at a peak range selector, a peak range of the smoothened EEG signals;
forming, at an artefact channel former, a pseudo artefact channel;
enabling, at a discriminative learner, discriminative learning; and
removing, at an artefact remover, ocular artefacts from the raw EEG signals.

10. The method of claim 9, further including:
initiating, at a signal correction module, signal correction of the raw EEG signals.

11. The method of claim 9, wherein the signal smoothener, the peak amplitude calculator, the peak range selector and the artefact channel former are integrated in an ocular artefact extraction module.

12. The method of claim 9, wherein the discriminative learner, and the artefact remover are integrated in a regularization optimization module.

13. The method of claim 9, wherein the signal smoothener relies on a moving average filter.

14. The method of claim 9, wherein the peak amplitude calculator determines a maximum relative amplitude of peaks of the smoothened EEG signal.

15. The method of claim 14, wherein separation of the ocular artefacts using the maximum relative amplitude of peaks enables separation of different ocular movements.

16. The method of claim 9, wherein the discriminative learner uses oscillatory correlation.

17. A non-transitory computer readable storage medium embodying thereon a program of computer readable instructions which, when executed by one or more processors of a signal processing device, cause the signal processing device to carry out a method for real-time discriminate ocular artefact removal from EEG signals, the method embodying the steps of:
smoothening, at a signal smoothener of the signal processing device, raw EEG signals;
calculating, at a peak amplitude calculator of the signal processing device, peak amplitudes of smoothened EEG signals;
selecting, at a peak range selector of the signal processing device, a peak range of the smoothened EEG signals;
forming, at an artefact channel former of the signal processing device, a pseudo artefact channel;
enabling, at a discriminative learner of the signal processing device, discriminative learning; and
removing, at an artefact remover of the signal processing device, ocular artefacts from the raw EEG signals.

18. The storage medium of claim 17, the method further embodying the step:
initiating, at a signal correction module of the signal processing device, signal correction of the raw EEG signals.
